(19) United States
(12) Patent Application Publication
(10) Pub. No.: US 2022/0222970 A1
(43) Pub. Date: Jul. 14, 2022

(54) OPHTHALMOLOGIC DEVICE WITH IMAGE STORAGE

(71) Applicant: Haag-Streit AG, Köniz (CH)

(72) Inventors: Frank ZUMKEHR, Zollikofen (CH); Jörg BREITENSTEIN, Zollikofen (CH)

(73) Assignee: Haag-Streit AG, Köniz (CH)

(21) Appl. No.: 17/613,126

(22) PCT Filed: May 23, 2019
(86) PCT No.: PCT/EP2019/063348

§ 371 Date: Nov. 22, 2021

(51) Int. Cl. G06V 40/18; G06V 20/40; G06V 20/69; A61B 3/135; A61B 3/14; A61B 3/00

(52) U.S. Cl. CPC G06V 40/197; G06V 20/40; G06V 40/193; A61B 3/0025; A61B 3/135; A61B 3/14; G06V 20/698

(57) ABSTRACT

An ophthalmologic device includes a microscope, an illumination system, a camera positioned to record an image through the microscope, and a storage device. When examining an eye, the camera is operated to continuously record a series of images. The images are stored in the storage device, each one with attributed imaging parameters describing the recording conditions of the image. When the examiner wants to retrieve images taking under examining conditions similar to the one presently used, the device is able to automatically retrieve the closest matches from the storage device. This allows to record, in the background, a large number of images documenting an eye's history and to retrieve them efficiently.

### TECHNICAL FIELD

The invention relates to an ophthalmologic device for examining an eye as well as to a method for operating an ophthalmologic device for examining an eye.

### BACKGROUND ART

In ophthalmology, a patient's eye is investigated by means of a device having a microscope. Modern devices comprise cameras that allow to record images viewed through the microscope. They also comprise a storage device for storing the images.

JP 2016209453 describes a device where some parameters under which the images are taken are recorded for documentation.

### DISCLOSURE OF THE INVENTION

The problem to be solved by the present invention is to provide a device and method of the type mentioned above that allow a versatile analysis of the eye.

This problem is solved by the device and method of the independent claims.

Accordingly, the device for examining an eye comprises at least the following elements:
A microscope: The microscope comprises a lens system suitable for obtaining and magnifying an image of the eye.
A camera: The camera is positioned to record an image through the microscope.
A storage device: The storage device is adapted and structured for storing at least the following information:

a) A plurality of images from the camera, i.e. recorded by the camera.

b) Attributed imaging parameters for these images. The “attributed image parameter(s)” for a given image is/are descriptive (i.e. provide information on) of at least one recording condition of the given image.
A control unit having a search unit: The search unit is adapted and structured to retrieve, from the storage device, one or more matching images given at least one “desired imaging parameter”.

In another aspect, the invention is implemented as a method for operating an ophthalmologic device for examining an eye, wherein the ophthalmologic device comprises a microscope, a camera positioned to record an image through the microscope, and a storage device as mentioned above. The method comprises at least the following steps:
Recording a plurality of images by means of the camera.
Storing the images: The images are stored in the storage device of the device.
Storing attributed imaging parameters for said images: The attributed imaging parameters are also stored in said storage device. As mentioned, the “attributed image parameter(s)” for a given image is/are descriptive (i.e. provide information on) of at least one recording condition of the given image.
Retrieving, from said storage device, one or more matching images given at least one desired imaging parameter.

In such a device and method, it is possible to provide one or more “desired imaging parameters” and then to search the stored images in the storage device based thereon. Hence, it becomes possible to search for images that were recorded under given imaging parameters (or parameters similar to them).

Advantageously, the device may comprise at least one current state monitor for determining at least one “current imaging parameter” of the device. This state monitor may e.g. be connected to at least one detector for detecting a setting of the device, and/or it can monitor the movement of actuators in the device and/or it can process the image recorded with the camera.

This e.g. allows to automatically use said current imaging parameter(s) as an attributed image parameter for an image recorded by the camera. In this case, the control unit may be adapted and structured to generate the “attributed imaging parameter(s)” from the current imaging parameter(s).

Also, the device can be adapted to use the current imaging parameter(s) to search the storage device for images that match them, at least to some degree. In this case, the search unit may be adapted and structured to generate the “desired imaging parameter(s)” from the current imaging parameter(s).

In one aspect, the method may comprise the following steps to be carried out during an examination of the eye:
Changing the settings of the device from a first to a second state by changing the current imaging parameters of said device while recording a series of images: For example, the examiner may zoom in various parts of the eye in order to find features of interest.
Automatically attributing, using said changing current imaging parameters, attributed imaging parameters to the series of images and storing said images and their attributed imaging parameters in said storage device. In other words, imaging parameters are attributed to the series of images and the result is automatically stored, thereby generating a record of the eye for different imaging parameters.

This allows generating a rich record of the state of the eye for differing imaging parameters at a given point in time. This record may later be recalled. For example, if the examiner detects a feature of interest in a given part of the eye in a future examination, she/he can retrieve earlier images of the same part in order to examine if that feature was present in the past.

It must be noted that the control unit of the device may be adapted to carry out the method steps of the invention by being programmed to do so. Hence, any method steps can also be formulated as the control unit being adapted to carry out said method steps.

In an advantageous embodiment, the device can comprise a slit lamp microscope.

### BRIEF DESCRIPTION OF THE DRAWINGS

The invention will be better understood and objects other than those set forth above will become apparent when consideration is given to the following detailed description thereof. This description makes reference to the annexed drawings, wherein:

FIG. 1 shows a lateral view of a slit lamp microscope,

FIG. 2 shows a top view of the microscope (with the slit lamp arm pivoted in respect to the microscope's optical axis),

FIG. 3 shows a block circuit diagram of the device,

FIG. 4 shows the steps in a typical examination, and

FIG. 5 shows an example of a user interface as displayed on a screen of the device.

### MODES FOR CARRYING OUT THE INVENTION

Device

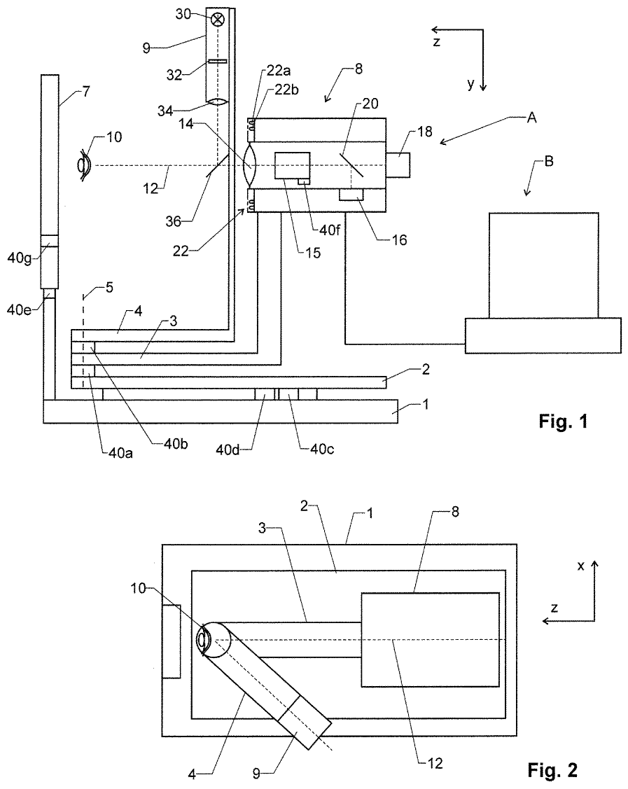

FIGS. 1 and 2 show an embodiment of a device based on a slit lamp microscope.

The shown device comprises an optical apparatus A and a computer B.

Optical apparatus A has a base 1 resting e.g. on a desk, a horizontally and vertically displaceable stage 2 mounted to base 1, a first arm 3, and a second arm 4.

The arms 3 and 4 are mounted to stage 2 and pivotal about a common vertical pivot axis 5.

Advantageously, arms 3 and/or 4 are manually operated, i.e. their angular position is changed manually, and they are not equipped with electric actuators. They may, however, also be provided with electric angular actuators to operate them automatically.

The device may further include a headrest 7 mounted to base 1 for receiving the patient's head.

Arm 3 carries a microscope 8, and arm 4 carries a first illumination source 9.

First illumination source 9 may e.g. be a conventional slit lamp as known to the skilled person, adapted to project a slit-shaped light beam onto the eye 10 to be examined.

Microscope 8 has an optical axis 12. It may comprise an entry objective 14, which projects an image of eye 10 onto a camera 16 and/or an eyepiece 18.

Microscope 8 may be provided with changeable zoom optics 15 for changing the optical magnification. Changeable zoom optics 15 may include continuously changeable zoom optics or stepwise changeable zoom optics (e.g. implemented as a Galilean optical system).

For quantitative measurements, the device advantageously is equipped with camera 16, while eyepiece 18 is optional. A beam splitter 20 may be arranged to spilt light between these components.

A plurality of microscope light sources 22a, 22b may be arranged on microscope 8 and movable together with it. They form a second illumination source 22. Advantageously, they are located around entry objective 14 and/or on a side of microscope 8 that faces eye 10.

Advantageously, the microscope light sources 22a, 22b are LEDs. They may, however, also be other types of light sources, e.g. semiconductor lasers.

Advantageously, the microscope light sources 22a, 22b may include infrared light sources 22a with a wavelength of at least 700 nm as well as visible light sources 22b with a shorter wavelength, e.g. a wavelength of less than 500 nm. Alternatively, the visible light sources 22b may e.g. emit green, red, or white light.

While first illumination source 9 is pivotal in respect to microscope 8, second illumination source 22 is fixed in respect to microscope 8.

First illumination source 9 comprises a light source 30, a modulator 32 and imaging optics 34.

Light source 30 can e.g. comprise several units emitting different wavelengths, e.g. in the red, green, blue, and infrared range of the optical spectrum. These units can be controlled separately in order to change the color of light source 30.

Modulator 32 is a spatial light modulator defining the cross section of the beam generated by first illumination source 9. It may e.g. be one of the solutions described in U.S. Pat. No. 5,943,118, such as a liquid crystal display or a controllable micro-mirror array.

Imaging optics 34 projects the light from modulator 32 onto the anterior surface of eye 10, e.g. via a mirror 36 mounted to arm 4.

Illumination source 9 can be arranged above or below mirror 36.

The device further comprises a control unit. In the present embodiment, said control unit is implemented in part in optical device A, e.g. as a microprocessor, and in part in computer B remote from optical device A. This will be described in more detail below.

The device may further comprise a number of detectors:
A first detector 40a may be provided for determining the angular position of first arm 3, i.e. the angle of the microscope's optical axis 12 in respect to the z-axis as shown in FIG. 2.
A second detector 40b may be provided for determining the angular position of second arm 4 in respect to the z-axis (or in respect to first arm 3).
A third detector 40c may be provided for determining the distance between microscope 8 and the eye 10. In the embodiment of FIG. 1, third detector 40c is shown as a detector, e.g. a magnetic position detector, adapted to measure the z-position of stage 2 in respect to base 1. From this position, as well as from the angular position of arm 3, the distance to the eye can be estimated. Alternatively, though, third detector 40c may e.g. be a counter connected to a stepper motor used for displacing stage 2 in respect to base 1 along direction z. Or it may e.g. be adapted to carry out an optical measurement for determining the distance between microscope 8 and eye 10.
A forth detector 40d may be provided for determining the horizontal x-offset of the microscope's optical axis 12 in respect to the eye. In the embodiment of FIG. 1, fourth detector 40d is shown as a detector adapted to measure the x-position of stage 2 in respect to base 1. Alternatively, though, fourth detector 40d may e.g. be a counter connected to a stepper motor used for displacing stage 2 in respect to base 1 along direction x. Or it may e.g. be adapted to carry out an optical measurement for determining the offset between the microscope's optical axis 12 and the center of the eye, e.g. using image processing on an image recorded by camera 16.
A fifth detector 40e may be provided for measuring the vertical y-offset of the microscope's optical axis 12 in respect to the eye. In the embodiment of FIG. 1, fifth detector 40e is shown as a detector adapted to measure the y-position (vertical position) of headrest 7, which may e.g. be adjustable manually or electrically. If an electrical actuator is provided for moving headrest 7 in y-direction, fifth detector may e.g. also be a counter counting the steps of a stepping motor. Or it may e.g. be adapted to carry out an optical measurement for determining the offset between the microscope's optical axis 12 and the center of the eye, e.g. using image processing on an image recorded by camera 16.
A sixth detector 40f may be provided for determining the current magnification as adjusted in zoom optics 15.
A seventh detector 40g may be provided for determining the presence of a patient in headrest 7. It can e.g. be used to end the storage of the images and attributed parameters in case the patient moves away from the device.

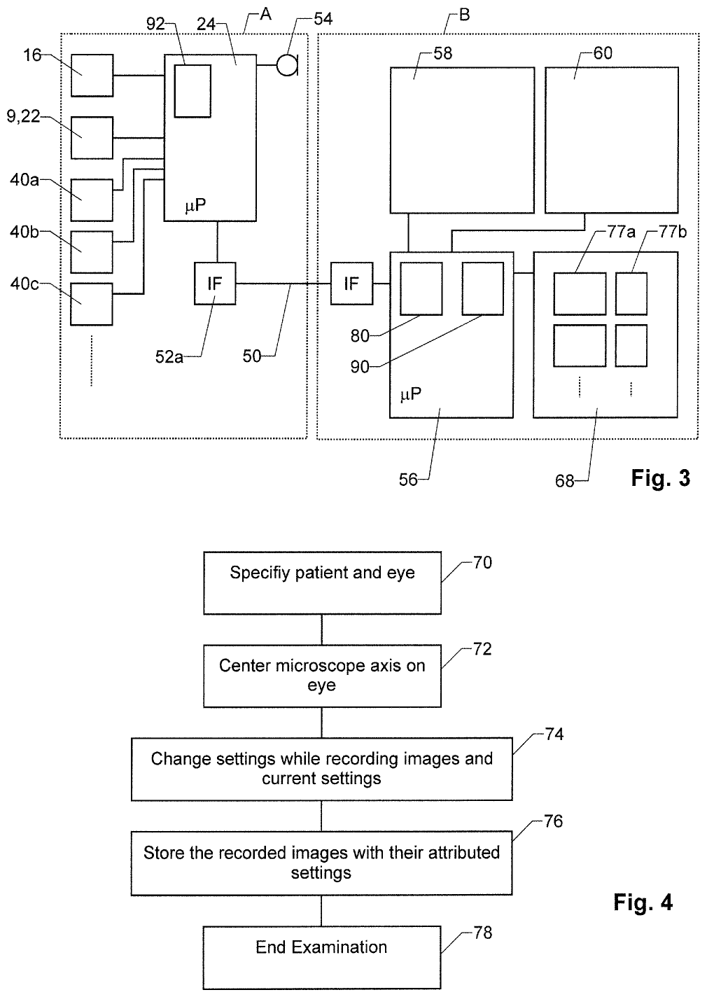

FIG. 3 shows a block circuit diagram of an embodiment of the device.

The components located in optical apparatus A and in computer B are enclosed with dotted lines labeled accordingly. A suitable interface 50 with interface circuits 52a, 52b connects these two parts. Interface 50 may be wire-bound or wireless.

Optical apparatus A comprises a control unit 24, such as a microprocessor with program control, which is connected to the various detectors 40a, 40b, etc. It is also connected to camera 16 for recording images and to the first and second illumination sources 9, 22 for controlling them.

Computer B also comprises a control unit 56, such as a microprocessor with program control, which is connected by means of driver circuitry to a display 58 as well as an input device 60. Input device 60 may e.g. be a keyboard and/or a touch-interface on display 58.

Computer B also comprises a storage device 68 for storing image and/or video data as well as other data as described in more detail below.

In the following, various scenarios while operating the device are described.

Device Operation

FIG. 4 illustrates the steps of a possible examination procedure.

In a first step 70, the examiner specifies the client being examined by entering a unique specifier into the device, e.g. by means of input device 60. This specifier may e.g. be a unique patient ID.

The examiner may also enter an identifier descriptive of the examination to be carried out.

Also, the examiner enters the eye to be examined, i.e. if he is about to examine the left or right eye. Alternatively, this information may be derived from the x-position of the microscope.

The device, e.g. computer B, will retain this information in its storage, e.g. by storing the patient ID, an examination specifier, and a left-right-eye indicator.

In a next step 72, the device may optionally be centered on the patient's eye. For example, the examiner can view the image recorded by microscope 8, e.g. through eyepiece 18 or as a life image of camera 16 on display 58, and adjust the microscope along the directions x and y until the eye's pupil is in its center. Also, the optical axis 12 of microscope 8 is brought into its angular center position, i.e. arm 3 is pivoted to align optical axis 12 with direction z.

Once this position is established, the examiner confirms proper alignment of the device by e.g. operating a control on optical apparatus A or computer B.

Starting from this moment, the device knows how microscope 8 is arranged in respect to the eye.

The device will now start to automatically record a series of individual images, e.g. a video feed, by means of camera 16.

Concurrently, the examiner will change the settings of the device in order to investigate one or more specific parts of the eye, step 74. For example, the examiner may offset the microscope along x, y, and/or z, change the viewing angle of the microscope, and/or change its magnification factor.

The device monitors and records these changes of the settings, i.e. it determines the “current imaging parameters”, e.g. in control unit 24. The current imaging parameters are sent to computer B together with the series of images, such that a set of imaging parameters can be attributed to each image.

Computer B stores the images and their “attributed imaging parameters” in storage device 68, step 76.

In the course of the examination, the examiner may explicitly chose to select some images, e.g. for a report, by entering a command in optical apparatus A or computer B. However, the device will not only store these selected images, e.g. marking them as “selected”, but the whole series of images for later retrieval.

FIG. 3 shows, schematically, the series of images 77a together with their attributed imaging parameters 77b in storage device 68.

When examination is complete, step 78, the examiner may specify this, e.g. again by means of input device 60. At this point, the automatic recording of images in storage device 68 may be terminated.

Hence, in the course of an examination, the device records a large number of images and stores them with their attributed imaging parameters in storage device 68, together at least with the patient ID.

Hence, in more general terms, the present method may contain the steps of
Determining a zero-position of microscope 8 in respect to the eye: This allows establishing a known position of microscope 8 in respect to the eye. This step can e.g. be carried out by centering optical axis 12 on the eye or by tracking the eye's periphery and e.g. statistically calculating the center of the eye therefrom.
Moving microscope 18 in relation to the zero-position by and x- and/or y-offset. Such movements can be monitored to determine the new current settings.
Using the x- and/or y-offset of microscope 8 as attributed imaging parameter(s) for images being recorded.

This allows to store, for every image, the relative location of the optical axis 12 in respect to the eye.

In another aspect, the method comprises at least the following steps:
Changing the device's settings from a first to a second state by changing the current imaging parameters of the device while recording a series of images: For example, as described above, the microscope may be offset or pivoted and/or its magnification factor may be changed.
Attributing, using the changing current imaging parameters, “attributed imaging parameters” to the images and storing the images and their attributed imaging parameters in storage device 68.

In this way, the device automatically stores a record of a large number of images, taken for N different imaging parameters in storage device 68. Advantageously, the number N is much larger than 1, e.g. 10 or more, during a single examination.

The images in storage device 68 may be stored as individual images. Alternatively, they may be stored as one or more video sequences, with at least some of the images stored as single frames of these video sequences, which may be a more compact form of storage.

For any such video sequence, the attributed image settings may change between frames. Hence, advantageously, storage device 68 holds, for at least some of the video sequences, parameter sequences describing how the attributed imaging parameters of the images change over said video sequence.

Image Retrieval

The device is equipped with a search unit 80, which is shown schematically as a functional block in FIG. 3. Search unit 80 is e.g. implemented as software run my computer B and forms part of control unit 56.

As mentioned above, search unit 80 is adapted and structured to retrieve, from storage device 68 and given at least one “desired imaging parameter”, one or more matching images.

For example, the examiner may see a feature of interest in the eye during examination and be interested to see older recordings of the same part of the eye, e.g. in order to view how an abnormality has developed over time. He then can use search unit 80 to retrieve older records of the same part of the eye.

To do so, he may e.g. use the current imaging parameters of the device, such as the current position of the camera and the current zoom factor, and automatically transfer them to search unit 80, which then searches storage device 68 for older images with the same or similar attributed imaging parameters.

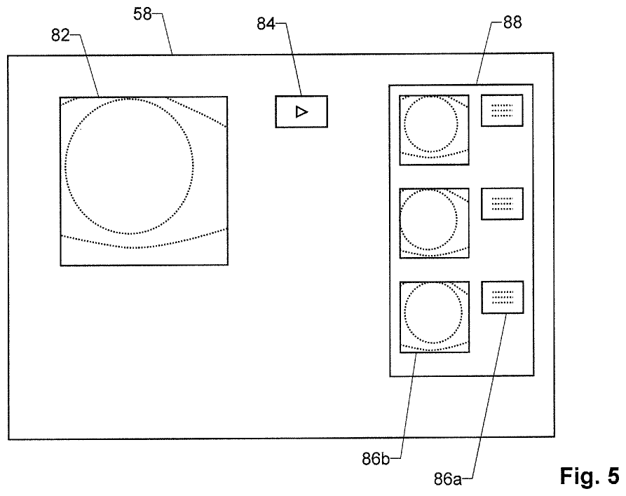

FIG. 5 shows an example what is displayed on display device 58 during such an operation. Part 82 shows the current image as seen through camera 16. Further, there is an interface element or key 84 for activating search unit 80. When interface element 84 is operated, the current imaging parameters are transferred to search unit 80, and search unit 80 browses storage device 68 for one or more close matches.

When such matches are found, the corresponding images 86a may e.g. be shown in a part 88 of display device 58, each of them with additional information 86b. Such additional information may e.g. be a time of recording of the image as well as, optionally, one or more of its attributed imaging parameters.

In the above example, the “desired” imaging parameters fed to search unit 80 are at least some of the current imaging parameters of the device.

Alternatively, or in addition thereto, the desired imaging parameters fed to search unit 80 may be generated as follows:
The examiner may enter them explicitly, e.g. in terms of an offset along directions x and/or y.
The examiner may indicate a part of the eye by using a descriptive search tell, such as “upper left quadrant”, “lower half”, “eye ground”, “lens”, “pupil”, “iris, limbus, or “Caruncula lacrimalis”.

The device may also comprise an image processor 90, which is shown as a functional unit in FIG. 3. Image processor 90 is e.g. implemented as software run my computer B and forms part of control unit 56.

Image processor 90 is able to identify, in an image recorded by camera 90, the subsection of the eye shown therein, e.g. it can recognize the “scene” visible in the camera. For example, given an image as shown in part 82 of FIG. 5, image processor 90 may identify
the coordinates of the center of the pupil, and
the radius of the iris.

These parameters, termed “subsection description”, describe the part of the eye visible in the image. As such, they are imaging parameters as mentioned herein. This subsection description can e.g. be used for the following applications:

a) It can be stored as attributed imaging parameters (or parts of the attributed imaging parameters) with the image they have been obtained from.

b) It can be fed to search unit 80 as “desired imaging parameters” in order to search storage device 68.

Hence, in more general terms, the method may comprise the following steps:
Analyzing at least part of the images recorded by camera 16 for automatically detecting the subsection of an eye visible in each image.
Generating a subsection description descriptive of said subsection.
Storing the subsection description with the image as attributed imaging parameter and/or using the subsection description as at least part of the desired imaging parameters to be fed to search unit 80.

Image processor 90 may operate concurrently with the recording of the images by means of camera 16 and feeding them to storage device 68.

Alternatively, the images can first be stored in storage device 68 and image processor 90 may process them at a later time. This provides more time and requires less computing power for processing and properly indexing the images.

Imaging Parameters

As mentioned, the invention relates to the use of imaging parameters of the device for storing these parameters together with the images (attributed imaging parameters) as well as for searching images (desired imaging parameters) as well as for describing the current setup and use of the device (current imaging parameters).

These imaging parameters may include one or more of the following parameters:
The viewing angle of microscope 8 (i.e. the angle between optical axis 12 and direction z in FIG. 2, e.g. as determined by detector 40a),
The x- and/or y-offset of optical axis 12 of microscope 8 in respect to a zero-position of the optical axis. This zero-position may e.g. be the one defined in step 72 of FIG. 4 and may e.g. be determined by detector 40d or 40e.
The distance of microscope 8 from the eye. This distance may e.g. be determined by detector 40c.
At least one setting of the illumination system 9, 22 of the device (see below).
The zoom setting of the microscope, which may e.g. be detected by detector 40f.
The aperture setting of the microscope if the microscope has an adjustable aperture.
A filter setting of the microscope if the microscope has a changeable spectral filter. Such a filter may e.g. be a changeable physical filter inserted between the eye and camera 16. Or it may be a numeric filter filtering the color image generated by camera 16.
A recording setting of camera 16. This setting may e.g. be the current gain and/or exposure time of the camera.
A left-right-eye indicator, i.e. information if the left or right eye is shown in the image, such as it was entered in step 70 of FIG. 4. This information may also be encoded from the device's x-position.
The patient ID uniquely identifying the patient.
A subsection description describing a subsection of an eye visible in a camera image, e.g. as determined by image processor 90 or derived from the zoom settings and/or the x- and/or y-offset.

As mentioned, the imaging parameters may include at least one setting of the illumination system 9, 22 of the camera, which comprises the first illumination system 9 (the slit lamp) and the second illumination system 22 (the light sources 22a, 22b) mounted to microscope 8. Such parameters may include:
A specification of the light sources used in the illumination system, i.e. a description of which light sources were on and which ones were off
A color setting of the illumination system: If light sources of different spectral properties are used, this may e.g. include a description of which of them were switched on or off. If spectral filters can be added to the illumination system, this may e.g. include a description of which filter(s) was/were used.
The geometry of the illumination system: This may e.g. include a description of the slit width used for a slit lamp, the orientation of the slit, and/or the position of the slit as projected onto the eye.
The angle setting of the illumination system: This may include the angular position of at least part of the illumination system. In the embodiment of FIGS. 1 and 2, this may e.g. be the angular setting of the slit lamp illumination system 9 as detected by second detector 40b.
The brightness setting of said illumination system. This describes the brightness set for the illumination system.

In order to determine the current imaging parameters, the device comprises a current state monitor 92, which may be incorporated in optical apparatus A, e.g. as a part of the software of control unit 24. Current state monitor 92 is able to determine the current imaging parameters of the device. It may do so by cooperating with the detectors 40a, 40b . . . . In addition thereto, or alternatively thereto, it may also be able to determine at least part of the current imaging parameters by monitoring the state of the device, e.g. the state of the stepper motors or other actuators in the device that change the settings, e.g. by monitoring actuators for displacing stage 2 in respect to base 1. It may also cooperate with image processor 90 for extracting at least part of the current imaging parameters from an image taken by camera 16.

Matching Imaging Parameters

The algorithm used by search unit 80 for identifying the images whose attributed imaging parameters best match the desired imaging parameters as well as for ranking them may depend on the type of imaging parameters. The following are some advantageous criteria assuming that the respective parameters are part of the imaging parameters:
a) The stored images may be filtered by patient ID.
b) The stored images may be filtered by left-right-eye indicator.
c) The stored images may be filtered or ranked depending on x- and y-offset. For example, only images where the absolute differences of x- and y-offset between the desired and attributed imaging parameters are within a certain threshold may be included.
d) The stored images may be filtered or ranked depending on the viewing angle of the microscope and/or depending on the illumination angle of illumination source 9 and/or depending on the mutual angle between the viewing angle of the microscope and the illumination angle of illumination source 9.
e) The stored images may be filtered or ranked depending on z-offset. For example, only images where an additional 90D lens was used. The slitlamp position is fare behind normal diagnose position.
f) The stored images may be filtered or ranked by zoom setting. This is particularly advantageous in combination with criterion c.
g) The stored images may be ranked by illumination parameters.
h) The desired parameters may e.g. be analyzed to calculate the desired region of the eye visible in the image. This region may be compared with the regions shown in the stored images to look for images having the largest mutual overlap with the desired region. This can e.g. be implemented using the subsection description mentioned above.

Search unit 80 may be configurable to use certain of these criteria and/or to ignore certain of these criteria.

Notes

In FIGS. 1 and 3, the device is shown to comprise an optical apparatus A and a computer B. It must be noted that this division is arbitrary. Part or all of the functionality of computer B may be incorporated in apparatus A, or the control functions of optical apparatus A may be completely implemented in computer B.

Also, part or all of the computing and storage functionality, and in particular storage device 68, may also be located at a remote site, such as on a remote server accessible e.g. through the internet.

To summarize, in one embodiment, the invention describes an ophthalmologic device that comprises a microscope 8, an illumination system 9, 22, a camera 16 positioned to record an image through said microscope, and a storage device 68. When examining an eye, camera 16 may be operated to continuously record a series of images. The images are stored in storage device 68, each one with attributed imaging parameters describing the recording conditions of the image. When the examiner wants to retrieve images taking under examining conditions similar to the one presently used, the device is able to automatically retrieve the closest matches from storage device 68. This allows to record, in the background, a large number of images documenting an eye's history and to retrieve them efficiently.

While there are shown and described presently preferred embodiments of the invention, it is to be distinctly understood that the invention is not limited thereto but may be otherwise variously embodied and practiced within the scope of the following claims.

1. An ophthalmologic device for examining an eye comprising
a microscope,
a camera positioned to record an image through said microscope,
a storage device adapted and structured for storing
a) a plurality of images from said camera and
b) attributed imaging parameters for said images, wherein the attributed imaging parameters of an image are descriptive of a recording condition of said image, and
a control unit having a search unit adapted and structured to retrieve, from said storage device, one or more matching images given at least one desired imaging parameter.

2. The device of claim 1, further comprising a current state monitor for determining at least one current imaging parameter of said device.

3. The device of claim 2, wherein said control unit is adapted and structured to generate the attributed imaging parameter(s) for an image from the current imaging parameter(s) of said device.

4. The device of any of claim 2, wherein said search unit is adapted and structured to generate said desired imaging parameter(s) from the current imaging parameter(s) of said device.

5. The device of claim 2, further comprising at least one detector connected to said current state monitor for determining at least one of said current imaging parameter(s).

6. The device of claim 1, wherein said storage device holds a plurality of video sequences, wherein at least part of said images are stored as frames of said video sequences.

7. The device of claim 6s wherein said storage device holds, for at least part of said video sequences, parameter sequences descriptive of changing attributed imaging parameters of the images in said video sequences.

8. A method for operating an ophthalmologic device for examining an eye, wherein said ophthalmologic device comprises
a microscope,
a camera positioned to record an image through said microscope, and
a storage device,
said method comprising:
recording a plurality of images by said camera,
storing, in said storage device, said images,
storing, in said storage device, attributed imaging parameters for said images, wherein the attributed imaging parameters of an image are descriptive of a recording condition of said image, and
retrieving, from said storage device, one or more matching images given at least one desired imaging parameter.

9. The method of claim 8, comprising of determining at least one current imaging parameter of said device.

10. The method of claim 9, comprising generating the attributed imaging parameter(s) for an image from the current imaging parameter(s).

11. The method of claim 9, comprising generating said desired imaging parameters from the current imaging parameter(s).

12. The method of claim 8, comprising:
determining a zero-position of said microscope in respect to said eye,
moving said microscope relative to said zero-position by and x- and/or y-offset,
using said x- and/or y-offset as imaging parameter(s).

13. The method of claim 8, comprising:
analyzing at least part of said images for automatically detecting a subsection of an eye visible in each image,
generating a subsection description descriptive of said subsection, and
storing said subsection description with the image as attributed imaging parameter and/or using said subsection description as at least part of said desired imaging parameters.

14. The method of claim 8, comprising:
changing settings of the device from a first to a second state by changing current imaging parameters of said device while recording a series of images, and
automatically attributing, using said changing current imaging parameters, attributed imaging parameters to said images and storing said images and their attributed imaging parameters in said storage device.

15. The method of claim 8, wherein said imaging parameters comprise at least one of:
a viewing angle of said microscope,
an x- and/or y-offset of an optical axis of said microscope in respect to a zero-position of said optical axis,
a distance of said microscope from said eye,
a setting of an illumination system of said device,
a zoom setting of said microscope,
an aperture setting of said microscope,
a filter setting of said microscope,
a recording setting of said camera,
a left-right-eye indicator,
a patient ID,
a subsection description descriptive of a subsection of an eye visible in a camera image.

16. The method of claim 15 wherein the setting of said illumination system comprises at least one of:
a specification of light sources used in the illumination system,
a color setting of said illumination system,
a geometry, in particular a slit width, slit orientation, and/or slit position, of said illumination system,
an angle setting of said illumination system,
a brightness setting of said illumination system.

17. The device of claim 1, wherein said imaging parameters comprise at least one of
a viewing angle of said microscope,
an x- and/or y-offset of an optical axis of said microscope in respect to a zero-position of said optical axis,
a distance of said microscope from said eye,
a setting of an illumination system of said device,
a zoom setting of said microscope,
an aperture setting of said microscope,
a filter setting of said microscope,
a recording setting of said camera,
a left-right-eye indicator,
a patient ID,
a subsection description descriptive of a subsection of an eye visible in a camera image.

18. The device of claim 17, wherein the setting of said illumination system comprises at least one of:
a specification of light sources used in the illumination system,
a color setting of said illumination system,
a geometry, in particular a slit width, slit orientation, and/or slit position, of said illumination system,
an angle setting of said illumination system,
a brightness setting of said illumination system.

19. A method for operating an ophthalmologic device for examining an eye, wherein said ophthalmologic device comprises:
a microscope,
a camera positioned to record an image through said microscope, and
a storage device,
said method comprising:
recording a plurality of images by said camera,
storing, in said storage device, said images,
storing, in said storage device, attributed imaging parameters for said images, wherein the attributed imaging parameters of an image are descriptive of a recording condition of said image,
retrieving, from said storage device, one or more matching images given at least one desired imaging parameter,
wherein said method further comprises:
analyzing at least part of said images for automatically detecting a subsection of an eye visible in each image,
generating a subsection description descriptive of said subsection, and
storing said subsection description with the image as attributed imaging parameter.

20. A method for operating an ophthalmologic device for examining an eye, wherein said ophthalmologic device comprises:
a microscope,
a camera positioned to record an image through said microscope, and
a storage device,
said method comprising:
recording a plurality of images by said camera,
storing, in said storage device, said images,
storing, in said storage device, attributed imaging parameters for said images, wherein the attributed imaging parameters of an image are descriptive of a recording condition of said image,
retrieving, from said storage device, one or more matching images given at least one desired imaging parameter,
wherein said method further comprises:
analyzing at least part of said images for automatically detecting a subsection of an eye visible in each image,
generating a subsection description descriptive of said subsection, and
using said subsection description as at least part of said desired imaging parameters.
